(19) United States
(12) Patent Application Publication
(10) Pub. No.: US 2026/0000826 A1
(43) Pub. Date: Jan. 1, 2026

(54) METHODS AND DEVICES FOR IMAGING BODY STRUCTURES

(71) Applicant: Femasys Inc., Suwanee, GA (US)

(72) Inventors: Kathy Lee-Sepsick, Suwanee, GA (US); Jeremy Sipos, Alpharetta, GA (US)

(21) Appl. No.: 19/252,694

(22) Filed: Jun. 27, 2025

Related U.S. Application Data
(60) Provisional application No. 63/666,128, filed on Jun. 29, 2024.

(51) Int. Cl. A61M 5/00; A61B 8/00; A61M 5/14; A61M 5/145; A61M 39/22; A61M 39/24

(52) U.S. Cl. CPC A61M 5/007; A61B 8/481; A61M 5/1408; A61M 5/1452; A61M 39/223; A61M 39/24; A61M 2005/006; A61M 2210/1425; A61M 2210/1433

(57) ABSTRACT

Disclosed herein are sonographic devices and methods of making and delivering a visualizable contrast medium composition, and methods for visualizing, diagnosing and treating structures in a subject.

### CROSS-REFERENCE TO RELATED APPLICATIONS

This patent application claims the benefit of U.S. Provisional Patent Application No. 63/666,128, filed Jun. 29, 2024, which is incorporated by reference herein in its entirety.

### BACKGROUND OF THE INVENTION

Using ultrasound methods for examining organs, ducts and cavities are well known and commonly used procedures. For example, hysterosonosalpingography or Sono HSG, uses ultrasound as the imaging modality. Ultrasound imaging with saline may be used for evaluation of the uterine cavity without assessment of fallopian tube patency. Tubal patency and tubal occlusion can be assessed only under ideal sonographic conditions, limiting its usefulness clinically. One method for enhancing the image quality of sonography, either by decreasing the reflectivity of the undesired interfaces or by increasing the backscattered echoes from the desired regions, is to use a contrast agent.

Conventional contrast agents have several disadvantages such as being expensive, difficult for ultrasound practitioners to use in the office setting if fluorographic contrast media is used, mixing steps such as shaking prior to injection to either mix the components or to generate bubbles, and a short shelf-life after mixing the contrast components.

Accordingly, devices and methods are needed for creating contrast agents that resolve the issues currently encountered. Particularly, methods and devices are needed for visualization of organ structure and function, such as visualization of the uterus and fallopian tubes.

### SUMMARY OF THE INVENTION

Disclosed herein are sonographic devices and methods for delivery of a contrast agent composition. The present disclosure comprises devices that are used in methods for making and using contrast media. Methods of the present disclosure comprise use of a device for generating a contrast medium. Methods of the present disclosure comprise use of a contrast medium, made with a disclosed sonographic device, for use in visualization procedures, such as ultrasound procedures for observing organs or bodily structures, for example, the uterus and fallopian tubes. Sonographic devices disclosed herein comprises a container assembly, two containers, that intakes the respective fluids (liquid and gas) into each of the respective liquid and gas containers (i.e., fills) simultaneously and at the same rate (speed and amount), and then creates and expels the contrast medium so that little to no individual fluid, such as liquid, exits the device's fluid port prior to the exiting or outward flow of the contrast medium. A container assembly comprises a first container for providing a liquid, such as saline, and a second container for providing a gas, such as air, which are used in making the contrast medium, and plungers for moving the liquid and gas. The contrast medium is then delivered to an organ or structure, or by use of a catheter or catheter assembly, which may be attached to the fluid port of the device.

Methods of the present disclosure comprise using a sonographic device disclosed herein to make an ultrasound visible contrast medium and to deliver an ultrasound visible contrast medium to a portion of a subject's body. Ultrasound devices can then be used to observe a structure in a body, such as a uterus only, or a uterus and its associated fallopian tubes. A method may further comprise use of a catheter or catheter assembly attached to the sonographic device and placement of a catheter delivery end in close proximity to the structure to be observed, and then providing the contrast medium to the structure. Methods of diagnosis and treatment are contemplated by the present disclosure, as the contrast medium provided may also comprise compounds or active agents for diagnosis and/or treatment of conduits and structures of a subject, which may or may not be observed by sonography. Methods of the present disclosure aid in the reproducibility of the methods of visualization and comparative results therefrom.

### BRIEF DESCRIPTION OF DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings. Alternative configurations of elements shown herein can be understood from the disclosure and figures herein.

FIG. 1 shows a drawing of the exterior of an exemplary disclosed device.

FIG. 2 shows a drawing of the interior of an exemplary disclosed device with the top part of the casing removed.

FIG. 3 shows a drawing of the interior of an exemplary disclosed device with the entire casing removed.

FIG. 4 shows a drawing of the components of an exemplary disclosed device.

### DETAILED DESCRIPTION OF THE INVENTION

Disclosed herein are devices that make and deliver a contrast medium for methods of diagnosis and treatment of a subject's organs or structures. Further disclosed are contrast medium compositions, and systems and kits comprising disclosed devices and compositions.

In an aspect, disclosed sonographic devices allow for the entry of gas and liquid into their respective gas and liquid containers to occur simultaneously and at the same rate. The gas and liquid are drawn into the device simultaneously, in that when the container plungers are moved proximally, both gas and liquid concurrently and simultaneously enter the device. Also, a disclosed device's containers fill simultaneously and at the same rate, meaning that the fluids, e.g., liquid and gas, enter the device and move through fluidly connected conduits, and fill the containers, illustrated herein as syringes, in substantially the same amount in substantially the same time period (rate of fill of containers). In a device, the plungers for both the air and liquid containers are moved simultaneously. For example, if the air container plunger is moved proximally to draw air into the device and positioned at 3 mL, showing 3 mL of air in the air container, then at the same time point from movement of both plungers, the liquid container plunger is also positioned at 3 mL and there are 3 mL of liquid in the liquid container. In contrast, in previous devices that have containers comprising syringes and plungers, though air and liquid may enter the device simultaneously, air enters the device and fills its respective container much faster than the liquid fills its respective container, even though the plungers for each container are moving proximally at the same rate (speed and distance). Thus, when both the air and liquid plungers have moved to each's most proximal position, the air container is full and the liquid container is still slowly filling with liquid. In previously known such devices, it has been found that it takes longer for the liquid container to fill with liquid, thus the liquid container and the air container are not filling at the same rate. The more slowly filling liquid container may lead to uneven amounts of gas and liquid in the resulting contrast medium if the operator does not correct for the time lag in filing both containers.

The presently disclosed sonographic device may achieve this simultaneous and substantially identical rate of filling both syringe containers by one or more of the following. A disclosed device may have liquid container fluid connections that are larger in diameter than are the air container fluid connections. A disclosed device may have similarly sized fluid connections for both the gas and liquid containers, but the gas fluid connections may be longer than the liquid fluid connections. A disclosed device may have similarly sized fluid connections for both the gas and liquid containers, but the gas fluid connections may be longer and may be more curved than the liquid fluid connections, whereas the liquid fluid connections are maintained in a straight line position, so that the liquid is pulled into its container by a direct straight fluid connection pathway, and the gas container's fluid connections and gas pathway are more sinuous and roundabout. A disclosed device may have larger diameter liquid container fluid connections than does the gas container fluid connections, and may have gas fluid connections that are longer than the liquid fluid connections, and may have different fluid connections pathways so that the liquid pathway is straight and the gas pathway is curved and roundabout.

The presently disclosed sonographic devices are designed so that the fluid first exiting a device is the contrast medium composition, with its mixture of gas and liquid, with very little, if any, unmixed liquid exiting prior to the exit of the contrast medium composition. With both gas and liquid containers filled, the respective plungers are moved distally simultaneously, and at the same rate, to move both gas and liquid, to form the contrast medium composition which exits the device as the dual component (gas and liquid) contrast medium composition). In contrast, in previous devices that utilize containers comprising syringes and plungers, in mixing two components, e.g., a gas and a liquid, when moving the plungers simultaneously and at the same rate, to move the two components, gas and liquid, from their respective containers in a distal direction so as to mix the gas and liquid, the liquid precedes the gas so that the first fluid dispensed from the device is the liquid, which is then followed by the contrast medium of mixed gas and liquid. This initial delivery of only liquid may slow the observation of the body structures until the contrast medium is delivered to the body structures. Also, the initial delivery of only liquid means less contrast medium is made and delivered, so that larger containers are needed so there is enough liquid to mix with the gas to make the contrast medium.

The presently disclosed sonographic device may achieve this release and exit of substantially only contrast medium by one or more of the following. For example, a disclosed device has two one-way valves, or check valves. The one-way valves may have the same cracking pressures or different cracking pressures. For example, one-way valve 270 (see FIG. 2) may have a higher cracking pressure than does one-way valve 280. One-way valve 280 may have a very low cracking pressure so that when the gas plunger is moved in a distal direction only a very slight air pressure is needed to open one-way valve 280 and gas flows quickly through its tubing to then substantially simultaneously meet the liquid at Y-shaped tubing 290. Because the one-way valve 280 opens at a low pressure and allows air to move quickly through from the beginning movement of the plungers, the contrast medium composition is made rapidly and exits the device, without liquid exiting prior to the exit of the contrast medium.

The present disclosure is directed to sonographic devices having specific components that are effective in methods for making and delivery of contrast medium compositions to structures, such as organs or conduits in a subject, such as a mammalian body, for example, delivery to one or more mammalian fallopian tubes. As used in the various figures herein, like numbers are used to describe like elements.

Aspects of disclosed devices may be best understood by reference to the drawings, wherein like parts are designated by like numerals throughout. It will be readily understood that the components of disclosed devices as generally described and illustrated in the figures herein could be arranged and designed in a wide variety of different configurations. Thus, the following more detailed description of various embodiments, as represented in the figures, is not intended to limit the scope of the disclosure, but may be merely representative of various aspects. In some cases, well-known structures, materials, or operations are not shown or described in detail. While the various aspects of the disclosed devices are presented in drawings, the drawings are not necessarily drawn to scale unless specifically indicated.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component. The terms “abut” and “abutting” refer to items that are in direct physical contact with each other, although the items may not necessarily be attached.

The terms “distal” and “proximal” are given their ordinary meaning in the art. That is, the distal end of a medical device means the end of the device furthest from the operator during use. The proximal end refers to the opposite end, or the end nearest the operator during use. As specifically applied to a container assembly, the distal end of the container assembly refers to the end nearest the inlet/outlet port of the syringe barrel or nearest the fluid port of the device, and the proximal end refers to the opposite end, the end nearest the head of the plunger or the device's syringe plunger actuator. Further, it will be appreciated that the phrase “distal end” always refers to the end nearest the fluid port, even if the distal end is temporarily closer to the operator at one or more points in a procedure.

The disclosure may be further understood from the FIGs. provided herein. The present disclosure is directed to devices having specific components that are effective in methods for making and delivery of contrast medium compositions to structures, such as organs or conduits in a subject, such as a mammalian body, for example, delivery to one or more mammalian fallopian tubes. As used in the various figures herein, like numbers are used to describe like elements.

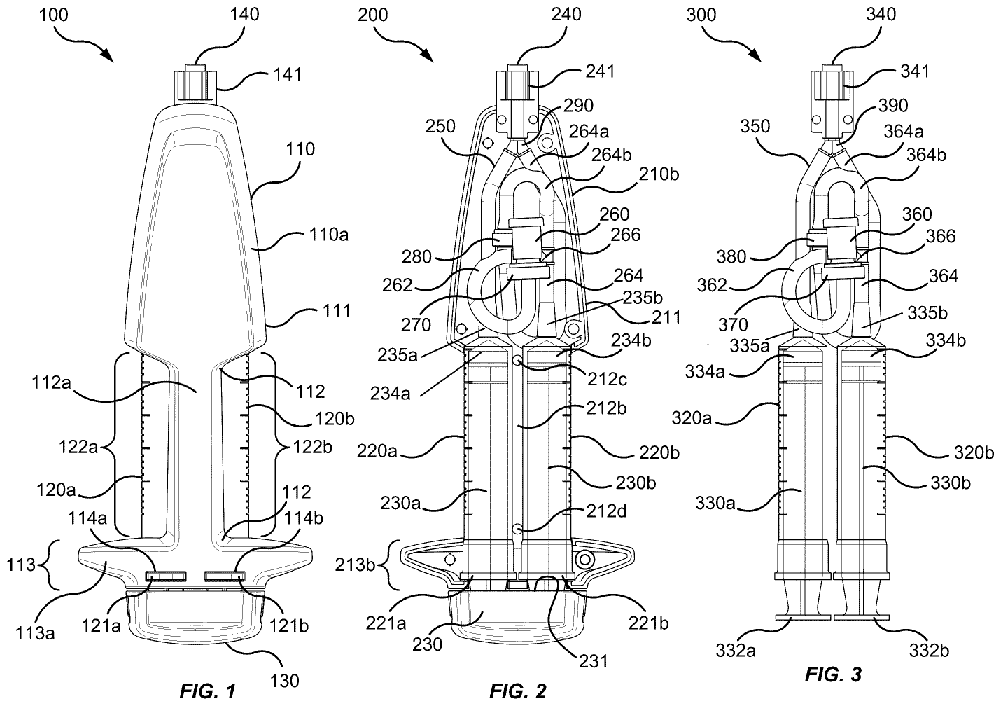

FIG. 1 illustrates an exemplary sonographic device 100. The top exterior side of casing 110, showing top casing side 110a of sonographic device 100. Casing 110 is formed by assembling top and back casing sides 110a and 110b, which is not shown, but is the same design and function as casing side 110a. FIG. 1 shows the exterior of an exemplary contrast agent delivery device 100 comprising,
a) casing 110 that is an outer casing enclosing interior elements of the device in enclosed portion 111, and forming connecting portion 112, and handle 113;
b) syringe bodies 120a and 120b; and
c) syringe plunger actuator 130.

As shown in FIG. 1, an exemplary disclosed sonographic device comprises casing 110 which is a casing that when the top and back casing sides, 110a and 110b (not shown) are joined, forms a wall that defines enclosed portion 111, connecting portion 112, and handle 113, formed from 113a and 113b (not shown). Casing 110 has top casing side 110a (and a back casing side 110b, the edge and interior of which is shown in FIG. 2 as 210b), and casing 110 may be assembled from two joined parts (110a and 110b) to form casing 110 and to define openings therein. Connecting portion 112 extends from enclosed portion 111 to handle 113, and has two components, 112a formed of front casing side 110a, and 112b formed of back casing side 110b (not shown) and the two components 112a and 112b of connecting portion 112 are snapped together at sites 212c and 212d located on each connecting portion and between syringe bodies 220a and 220b (See FIG. 2). The location of sites 212c and 212d are not limited to those shown in FIG. 2 but may be placed in other locations so that connecting portions 112a and 112b are maintained together. The exterior of syringe plunger actuator 130 is shown in FIG. 1.

Syringe body 120a and syringe body 120b are parallel to each other and each has a proximal flange 121a and 121b, respectively, that protrude from handle 113 through slots 114a and 114b which are both formed in each casing side 110a and 110b (not shown) in handle 113. Syringe bodies 120a and 120b each have markings 122a and 122b along each outward surface which indicate the amount of fluid contained with the syringe body or the position of the plunger(s), which are not shown in FIG. 1. Markings are known to be present on syringe bodies. As this device is designed to make and deliver small amounts of contrast agent composition, generally from about 0.2 mL to about 10 mL, each syringe body is relatively small, such as those shown in FIG. 1 which are 5 ml syringe bodies. Other syringe sizes are contemplated by the present disclosure, and are known to those of skill in the art.

Fluid port 140 is in fluid connection with syringe bodies 120a and 120b, respectively, and in some aspects serves as an inlet port for liquids and in other aspects, serves an exit port for contrast agent compositions. Fluid port 140 may comprise an attachment element 141, which may be for example, a known attachment element such as a luer lock component.

FIG. 2 shows sonographic device 200 with the front casing side (110a of FIG. 1) removed to illustrate interior elements and their arrangements and positions within the interior of casing 211, and are shown on the interior of back casing side 210b. Interior of handle 213b and connecting portion 212b are shown. Also shown are syringe bodies 220a and 220b, with their respective plungers, 230a and 230b. Each syringe body 220a and 220b, has syringe distal end 235a and 235b, respectively, which is shaped to provide an opening in the syringe body for fluid connection and attachment for fluidly connecting with respectively, liquid connecting tubing 250, or air connecting tubing 264. Each plunger has a proximal end 232a and 232b (not shown, see FIG. 3), which are fixedly held by syringe plunger actuator 230, and a distal end, 234a and 234b. Syringe flanges 221a and 221b are shown positioned in handle 213b. The exterior of syringe plunger actuator 230 is shown in FIG. 2, and syringe plunger actuator 230 is a hollow structure with its distal side 231 open to receive proximal end 232a and 232b (not shown) of each plunger 230a and 230b. When plunger actuator 230 is moved in a proximal direction, toward the device user, both plunger 230a and 230b move simultaneously in a proximal direction, and when plunger actuator 230 is moved in a distal direction, away from the user and toward fluid port 240, both plungers move simultaneously in a distal direction.

Syringe body 220a is in fluid connection with fluid port 240 via liquid connecting tubing 250 and Y-shaped tubing 290. Syringe body 220b is in fluid connection with air inlet port 260 through air inlet connecting tubing 262, tubing connection 266, and air connecting tubing 264, and with air exiting connecting tubing 264a and 264b, which are in fluid connection with fluid port 240 via Y-shaped tubing 290. Air inlet tubing 262 is in fluid connection with air connecting tubing 264 at tubing connection 266. Between air inlet port 260 and air inlet connecting tubing 262, and in fluid connection, is one way valve 270. Syringe body 220b is also in fluid connection with air connecting tubing 264, tubing connection 266, air exiting tubing 264b, air exiting tubing 264a and Y-shaped tubing 290. Between air exiting tubing 264b and air exiting tubing 264a, and in fluid connection, is one way valve 280. Attachment element 241, which may be for example, a known attachment element such as a luer lock component, is shown.

In FIG. 3, shows an exemplary sonographic device 300 in which both casing sides are removed and the syringe bodies 320a and 320b and each respective plunger, 330a and 330b, in addition to interior elements are shown. The proximal ends 332a and 332b are visible with the removal of plunger actuator 230, shown in FIG. 2. Distal ends 334a and 334b of plungers 330a and 33b are shown within syringe bodies 320a and 320b, respectively. The interior elements, discussed above, are also shown. Each syringe body 320a and 320b, has syringe distal end 335a and 335b, respectively, which is shaped to provide an opening in the syringe body for fluid connection and attachment for fluidly connecting with respectively, liquid connecting tubing 350, or air connecting tubing 364. Syringe body 320a is in fluid connection with fluid port 340 via liquid connecting tubing 350 and Y-shaped tubing 390 . . . . Syringe body 320b is in fluid connection with both air inlet port 360 through air inlet connecting tubing 362, tubing connection 366 and air connecting tubing 364, and also with air exiting connecting tubing 364a and 364b, which are in fluid connection with fluid port 340 via Y-shaped tubing 390. Air inlet tubing 362 is in fluid connection with air connecting tubing 364a at tubing connection 366. Between air inlet port 360 and tubing connection 366, and in fluid connection, is one way valve 370. Syringe body 320b is in fluid connection with air connecting tubing 364, tubing connection 366, air exiting tubing 364b, air exiting tubing 364a and Y-shaped tubing 390. Between air exiting tubing 364b and air exiting tubing 364a, and in fluid connection, is one way valve 380. Attachment element 341, which may be for example, a known attachment element such as a luer lock component, is shown.

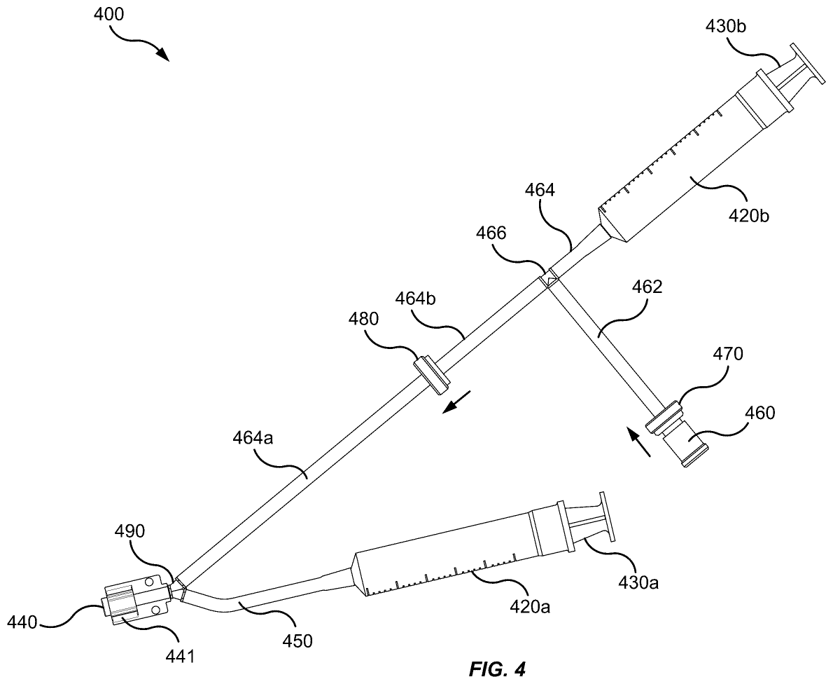

FIG. 4 shows an expanded version of the interior elements of a disclosed sonographic device 400. Syringe body 420a, with plunger 430a residing within, is in fluid connection with fluid port 440 via liquid connecting tubing 450 and Y-shaped tubing 490. In filling syringe body 420a with a liquid, fluid port 440 is in contact with the liquid, and as plunger 430a is moved in a proximal direction, liquid is drawn into and through fluid port 440 through Y-connecting tubing 490, through liquid connecting tubing 450 and into syringe body 420a. When plunger 430a is moved in a distal direction, liquid exits syringe body 420a, flows through liquid connecting tubing 450 to Y-shaped tubing 490. It is at Y-shaped tubing 490 that the liquid and air/gas interact and form the contrast medium composition. The contrast medium composition then exits fluid port 440. Y-shaped tubing 490 is in fluid connection with both syringe body 420a and syringe body 420b.

Syringe body 420b is in fluid connection with air inlet port 460 through air inlet connecting tubing 462, tubing connection 466, and air connecting tubing 464, and also with air exiting connecting tubing 464a, which is in fluid connection with fluid port 440 via Y-shaped tubing 490. Air inlet tubing 462 is in fluid connection with air connecting tubing 464 at tubing connection 466. Syringe body 420b is in fluid connection with air connecting tubing 464, tubing connection 466, air exiting tubing 464b, air exiting tubing 464a and Y-shaped tubing 490. Between air inlet port 460 and air inlet connecting tubing 462, and in fluid connection, is one way valve 470. In fluid connection with, and between air exiting tubing 464b and air exiting tubing 464a is one way valve 480. Attachment element 441, which may be for example, a known attachment element such as a luer lock component, is shown.

The tubing or fluid connections between syringe body 420b and fluid port 440 have been numbered so that the movement of air is understood. Air entering through air inlet port 460 is drawn into syringe 420b, by action of plunger 430b in a proximal direction, through one-way valve 470, through air inlet tubing 462, through tubing connection 466 and air connecting tubing 464, which are all in fluid connection. One way valve 470 is positioned to only allow air to enter through air inlet port 460 and for no air to exit through air inlet port 460. Air is provided from syringe 420b, by action of plunger 430b in a distal direction, so that air (gas) is moved out of syringe 420b, into air connecting tubing 464, tubing connection 466, air exiting tubing 464b, one-way valve 480, air exiting connecting tubing 464a and Y-shaped tubing 490. One-way valve 480 allows only air to flow through it from air exiting connecting tubing 464b and into air exiting connecting tubing 464a, and subsequently to Y-shaped tubing 490. Air cannot flow from air exiting connecting tubing 464a back into air exiting connecting tubing 464b. The present disclosure comprises methods and devices for making and using contrast medium for ultrasound or sonographic procedures of visualizing structures in subjects. Such structures may be present in the bodies of subject such as humans or animals, or may be inanimate structures. As discussed herein, the methods and devices may be used for ultrasound visualization of a uterus only, or a uterus and one or more fallopian tubes of a mammal. It is to be understood that the methods and devices are not limited to this application, but can be used in visualization of ducts or structures, whether in living beings or inanimate structures.

The present subject matter comprises devices for making a contrast medium composition. As used herein, contrast medium means a composition that is visible or visualizable by methods known to those skilled in the art, including but not limited to ultrasound, fluorography, radiography, or other detection methods, and the devices and methods disclosed herein will refer to a gas/liquid contrast medium and sonography for ease of understanding, which is not to be seen as limiting to the methods or devices herein. A method of the present disclosure comprises use of a contrast medium device for generating and delivering a contrast agent that is useful for sonographically observing organs or bodily structures, for example, the uterus and/or fallopian tubes. A method of the present disclosure comprises use of a contrast medium device for generating a fluid contrast agent or composition that is useful for sonographically observing organs or bodily structures, for example, at least one fallopian tube. Methods may also comprise diagnostic and therapeutic fluids used in disclosed devices for making compositions used in methods of diagnosis or treatments of bodily structures. To facilitate ease of understanding, the term “contrast agent” or “contrast medium” or “contrast medium composition” may be used interchangeably, and it is understood that each of these compositions may include one or more diagnostic and/or therapeutic agents (e.g., bioactive agents, compounds, molecules, elements and/or salts thereof) and may be intended for methods of diagnosis or treatment.

A contrast medium device comprises the components disclosed in a device herein, and optionally, may further comprise a catheter, or catheter assembly or device, fluidly coupled to the disclosed contrast medium device. In general, a disclosed device comprises two containers, each for containing a fluid. A fluid may be a liquid or a gas. A resulting contrast agent composition comprises a fluid, made from the fluids, liquid and gas. In an aspect, a disclosed device comprises a first container for a liquid, such as saline, and a second container for a gas, such as air. A disclosed device further comprises connection elements, such as tubing or fluid conduits, for providing a fluid to its respective container, and for providing each contained fluid from its respective container, and the created fluid contrast medium to the fluid port, and if present, to an attached catheter. A container comprises an opening which may function as an inlet port for fluid coming into the container, or as an outlet port for fluid exiting the container. For example, the container is a syringe body with a plunger positioned therein. The plunger is inserted through a larger opening in one end of the syringe, and a smaller opening, the inlet/outlet port, is at the opposite end of the syringe body so that movement of the plunger causes fluid to enter or exit the syringe body. Moving a plunger in a syringe provides a force upon the fluid contained within the syringe to move fluid into, or out of, the syringe. Activating or moving the plunger may be by a pump, or the hand of an operator. In an aspect, a disclosed device comprises an actuator for simultaneous movement of the two plungers, such that the syringe plungers move through the interior of the barrel of the syringes at the same rate, (i.e., speed and distance), through the interior of the respective syringes. The syringe plungers move at the same rate, (i.e., speed and distance), because the proximal ends of each plunger are maintained together, by an actuator. For example, see FIG. 2 plunger activator 230.

A disclosed device may further comprise fluid connections, which are fluid connecting tubing between elements that are in fluid connection with one another. Such fluid connections are achieved with known components which include, but are not limited to, conduits or tubing. A disclosed device may comprise fluid connections which provide the contrast medium composition to a catheter, catheter assembly or device, or directly to a structure to be visualized. A disclosed device may be in fluid connection with a catheter or catheter assembly by an attachment element at the fluid port. An attachment element is known in the art, such as a luer lock, and is used to attach the disclosed device to a catheter or catheter assembly. The catheter may comprise a single or double lumen catheter. A catheter may comprise one or more balloons as elements of a device. A catheter may comprise end structures, such as a balloon on the delivery end of the catheter, wherein the delivery end is distal from the contrast medium device and the attachment end is proximal to the contrast medium device, and may have attachment elements for attaching the catheter to a disclosed device. The catheter or catheter assembly may comprise other components such as a wire, sensors, cutting elements, retrieval elements such as clamps or pincers. A catheter may be an HSG catheter having a balloon located in a proximal portion of the catheter. Such catheters are known in the art and one skilled in the art can select an appropriate catheter for the intended procedure.

A disclosed device, in combination with a catheter or catheter assembly, may provide contrast medium substantially directly to a structure to be visualized. In an aspect of the present subject matter, for example, in direct delivery to the uterus, and or a uterus and at least one fallopian tube, the amount of contrast medium used may be small, such as less than 10 mL, less than 8 mL, less than 5 mL, less than 4 mL, less than 3 mL, less than 2 mL, less than 1 mL, less than 0.5 mL. The amount of contrast medium used may be an amount that is sufficient to provide an accurate visualization of the structure. The contrast medium may substantially fill the structure visualized, or may only be present in particular locations within the structure. A disclosed device, in combination with a catheter or catheter assembly, may deliver a contrast medium to one or more structures, such as to multiple cavities, organs or conduits that are in fluid connection with one another. The amount of contrast medium used may be an amount that is sufficient to provide an accurate visualization of the structure to be examined. The contrast medium may substantially fill the structure visualized, or may only be present in particular locations within the structure. A disclosed device and methods using a disclosed device allows for control of the contrast medium delivery, so that some or all of the contrast medium composition may be provided to a body structure. The flow of the contrast medium out of the device and/or out of a catheter, and to a body structure, may be controlled so that providing the contrast medium may be in a continuous flow or intermittent flow, such as providing some contrast medium, stopping the flow, providing contrast medium, stopping the flow, and so on. The containers of a disclosed device may be refilled one or more times during a procedure. The flow of contrast medium to the structure may be controlled automatically or by an operator. The rate of delivery of contrast medium may be controlled. The rate of delivery may be in a range from fast to slow, and is primarily controlled by the rate of movement of the plungers.

A method of the present disclosure comprises using a disclosed device and catheter or catheter assembly to provide contrast medium to the uterus for visualizing at least one fallopian tube, or visualization of at least a portion of the uterus and at least one fallopian tube. When the structure to be visualized is at least one fallopian tube, or a uterus and/or at least one fallopian tube, a disclosed device, in combination with a catheter may be used. A catheter having elements for preventing retrograde flow of fluid from the uterus may be connected to a disclosed device. Catheters with element(s) that prevent retrograde flow are known in the art, and it is within the skill of those in the art for selecting a catheter to attach to a disclosed device to use in methods taught herein. As used herein, a catheter assembly comprises a catheter and other components to form a device that aids in delivery of one or more fluids to a structure. For example, see U.S. Pat. No. D654,583, which is herein incorporated in its entirety for its teachings. A syringe may be used to inflate or deflate a catheter balloon. Syringes for inflating a catheter balloon are known in the art. A method disclosed herein may comprise use of a variably locking syringe for inflation/deflation of a catheter balloon. For example, a variably locking syringe is taught in U.S. patent application Ser. No. 18/437,667, which is incorporated in its entirety, for all of its teachings.

In an aspect, a method comprises attaching a catheter, such as a balloon catheter, to a disclosed device. In the method, the delivery end of the catheter is placed in the uterus, and optionally, the catheter comprises a structure to prevent retrograde flow into the cervix, for example, the structure, such as a balloon, is expanded, by optionally using a variably locking syringe, such as that taught in U.S. patent application Ser. No. 18/437,667. The contrast medium is generated by the actions of the disclosed device and the contrast medium is provided into and through the catheter and out into the uterus. The desired amount of contrast medium is provided and visualization techniques are initiated, and the movement of the contrast medium into the uterus is visualized, or at least a portion of the structure of the uterus is visualized, for example, by providing the contrast medium. Further, visualization of entry, transit and/or exit of the contrast medium in at least one fallopian tube may occur. If a fallopian tube is blocked, the contrast medium will not flow past the blockage but may flow to the contralateral tube. If not blocked, the contrast medium may flow into the peritoneal cavity. A disclosed device may be unattached from the proximal end of the catheter and the disclosed device may be refilled with more of the same types of fluids, as was used in the first delivery of contrast medium, or different fluids if desired. In using a disclosed device, once an amount of contrast medium is provided and the containers are depleted of fluids, the containers may be refilled one or multiple times so as to provide an effective amount of contrast medium to the structure and/or conduits of the subject.

In an aspect, a disclosed device functions and is operated in the following manner, as exemplified in FIG. 2. A method of making a contrast medium using a sonographic device disclosed herein comprises the following steps. Fluid port 240, of a disclosed device, is inserted into a liquid, such as a sterile liquid, for example, saline. The liquid may be in a container provided in a kit disclosed herein, or separately provided by an operator, such as a healthcare provider. If the device is in an initial position (or a final position), as shown in FIG. 2, with the plungers moved to the most distal position, to start the filling of the device, syringe plunger actuator 230 is moved in a proximal direction to move plungers 230a and 230b in a proximal direction at the same rate and same distance through the respective syringe bodies. Simultaneously, liquid is drawn into fluid port 240 and air or gas is drawn into air inlet port 260. As plungers 230a and 230b move proximally toward the most proximal positions, air (or gas) and liquid enter and respectively fill syringe bodies 220a and 220b at the same rate and in the same amount. If a gas other than surrounding air is desired, air inlet port 260 may comprise attachments elements, known to those of skill in the art, so that one or more gases may be provided to the device to be used to make a contrast medium composition. The attachment elements and gas connections are not shown in the figures herein, but are readily understood by those of skill in the art. For ease of understanding, the term air will be used, with the understanding that air also means one or more specific gases.

To fill the air and liquid containers, the plungers (not shown in FIG. 1) of syringe body 120a and 120b are positioned at the most distal position, with the plunger actuator 130 in closest proximity to handle 113. Action of device components can be understood from FIGS. 2-4. Proximal movement of syringe plunger actuator 230 moves syringe plunger 230a and 230b simultaneously and at the same rate (speed and distance) proximally through respective syringe bodies 220a and 220b. Fluid port 240 is immersed into or contacting a liquid. The liquid is drawn into fluid port 240, passing in a proximal direction through Y-shaped tubing 290 and liquid connecting tubing 250 to enter syringe body 220a. As can be seen in FIG. 2, liquid connecting tubing 250 is a substantially straight tubing connection between Y-shaped tubing 290 and the distal end of syringe body 230a. Air is drawn in through air inlet port 260, passing through one-way valve 270, through air inlet connecting tubing 262, through tubing connection 266, through air connecting tubing 264 and into syringe body 220b. As can be seen in FIGS. 2-3, the route for air from air inlet port 260 to syringe body 220b, is a curved and sinuous route through one-way valve 270, through air inlet connecting tubing 262, through tubing connection 266, through air connecting tubing 264 and into syringe body 220b. Air and liquid enter and respectively fill syringe bodies 220a and 220b at the same rate and in the same amount. Plungers 230a and 230b are moved by syringe actuator 230 in a proximal direction for the distance desired by the operator, for example, to the most proximal position to fill each syringe body 220a and 220b with its respective fluid.

In an aspect, to form a contrast medium composition, comprising liquid (e.g., saline) and gas (e.g., air), the following occurs. In an aspect, syringe bodies (containers) 220a and 220b have been completely filled with the respective fluids, and plungers 230a and 230b are at the most proximal position. Syringe actuator 230 is moved in a distal direction to simultaneously move both syringe plunger 230a and 230b at the same rate (speed and distance). Movement of syringe plungers 230a and 230b cause the respective fluids, liquid and air, to begin to exit and to continue exiting the respective containers, syringe bodies 220a and 220b. Liquid exiting syringe body 220a transits liquid connecting tubing 250 and Y-shaped tubing 290. Simultaneously and at the same rate (speed and distance) air exits syringe body 220b, transiting, in fluid connection, air connecting tubing 264, tubing connection 266, air exiting connecting tubing 264b, one-way valve 280, air exiting connecting tubing 264a to Y-shaped tubing 290.

At Y-shaped tubing 290, air and liquid meet and join substantially simultaneously to form the contrast medium comprising segments of air and liquid. Continued movement distally by syringe plungers 220a and 220b move the contrast medium from Y-shaped tubing 290 through and out fluid port 240. In an aspect, by action of plunger 230a in a proximal direction, liquid enters device 200 through fluid port 240, and through tubing connections is contained in syringe body 220a. In an aspect, by action of plunger 230b in a proximal direction, air enters device 200 through air inlet port 260, and through tubing connections is contained in syringe body 220b. In an aspect, by action of plunger 230a in a distal direction, liquid exits syringe body 220a, and through tubing connections is moved to Y-shaped tubing 290. In an aspect, by action of plunger 230b in a distal direction, air exits syringe body 220b, and through tubing connections is moved to Y-shaped tubing 290. By continued action of both plungers 220a and 220b distally moving simultaneously and at the same rate (speed and distance), contrast medium is formed from the liquid and air in Y-shaped tubing 290, and the contrast medium is moved through and out fluid port 240. Little to no individual and separate liquid or air exits fluid port 240.

One-way valve 280 opens at a low pressure, referred to as cracking pressure. Cracking pressure refers to the inlet pressure level at which the first sign of flow is present. It can also be described as a measure of the pressure differential between the inlet and outlet ports of the valve when flow is initially detected. Specifically, cracking pressure is the least differential pressure that the valve experiences during flow. One-way valve 280 has a cracking pressure of approximately from 0.014 to 0.087 lb/in² (psi), or approximately less than 0.087 lb/in². In an aspect, both one-way valves 280 and 270, each have the same cracking pressure of approximately from 0.014 to 0.087 lb/in² (psi), or approximately less than 0.087 lb/in². In an aspect, at this cracking pressure, the valve is completely open and air flows freely through the valve at the pressure provided by the distal movement of the respective plunger. In an aspect, one-way valves 280 and 270 may have different cracking pressures. In an aspect, the cracking pressure of valve 280 may exceed the cracking pressure of valve 270. In an aspect, the cracking pressure of valve 270 may exceed the cracking pressure of valve 280. Other aspects and arrangements may exist without departing from the scope of the present subject matter.

In an aspect, a catheter or catheter assembly is attached to attachment element 241 so that the catheter is in fluid connection with fluid port 240. Contrast medium exiting fluid port 240 enters the attached catheter and the contrast fluid is then provided to the desired structure. For example, contrast medium may enter a cavity, such as the uterus, and further enter and potentially transit, at least one fallopian tube, wherein visualization of the contrast medium within the structure can be conducted, such as by sonographic methods, and the one or more body structures are examined and diagnosed. For example, if the contrast medium comprises therapeutic or treatment compounds or molecules in a method of treatment, treatment may be provided to one or more structures, alone or in combination with visualization methods. The catheter or catheter assembly may comprise a single or double lumen catheter. The catheter may comprise end structures, such as a balloon on the delivery end of the catheter, wherein the delivery end is distal from the contrast medium device and the attachment end is proximal to the contrast medium device. The attachment end of the catheter may have attachment elements for attaching the catheter to a sonographic device disclosed herein. Attachment elements such as a luer lock, may be used to attach the catheter to a disclosed device, and attachment elements are known. The catheter may comprise other components such as a wire, sensors, cutting elements, retrieval elements such as clamps or pincers. Such catheters are known in the art and one skilled in the art can select an appropriate catheter for the intended procedure.

Methods of the present disclosure comprise providing a device disclosed herein for visualization, diagnosis or treatment of one or more body structures of a subject. In an aspect, a method may comprise delivery of a contrast medium to a body structure of a subject. Contrast medium may be provided indirectly or directly to a body structure. For example, contrast medium may be provided directly to a uterus, and by the flowing action of the contrast medium, the contrast medium is indirectly provided to one or more fallopian tubes in fluid connection with the uterus or the abdominal cavity or structures therein which is in fluid connection with one or more fallopian tubes. The contrast medium may be used to visualize or treat such direct or indirectly contacted tissues, structures or organs. The amount of contrast medium used may be an amount that is sufficient to provide an accurate visualization or treatment of the tissues, structures or organs. In an aspect, contrast medium from a disclosed device may be provided directly to a structure for visualization or treatment thereof. For example, a catheter or catheter assembly may be used to deliver contrast medium from a disclosed device directly to one or more fallopian tubes, without filling of the uterus, or visualizing or treating the uterus. This disclosure is not limited to the specific body structures noted herein as these are exemplary methods, and those of skill in the art can use disclosed devices for visualization or treatment of body structures of subjects or inanimate objects.

In an aspect, delivery of a contrast medium made by a disclosed device is controlled so that some or all of the contrast medium may be provided to a body structure. The flow of the contrast medium out of the device and/or out of a catheter, and to a body structure may be controlled so that providing the contrast medium may be in a continuous flow or intermittent flow, such as providing some contrast medium, stopping the flow, providing contrast medium, stopping the flow, and so on. The container(s) of a disclosed device may be refilled one or more times during a procedure. The flow of contrast medium to the structure may be controlled automatically or by an operator. The rate of delivery of contrast medium may be controlled. The rate of delivery may be in a range from fast to slow, and is primarily controlled by the rate of force applied to the component(s) for providing force upon the fluid contained within the container(s).

In an aspect, a disclosed method comprises using a disclosed device and a catheter or catheter assembly to provide contrast medium to the uterus for visualizing at least one fallopian tube, or visualization of at least a portion of the uterus and at least one fallopian tube. A catheter, or catheter assembly, having elements for preventing retrograde flow of fluid from the uterus may be connected to a disclosed device. Catheters with element(s) that prevent retrograde flow are known in the art, and it is within the skill of those in the art for selecting a catheter to attach to a disclosed device to use in methods taught herein.

In an aspect, a method may comprise use of a catheter, such as a balloon catheter, or a catheter assembly comprising a balloon catheter. In a method, the delivery end of a catheter is placed in the uterus, and optionally, the structure to prevent retrograde flow into the cervix is employed, for example, the balloon of a balloon catheter is expanded, by the use of a syringe, or a variably controlled syringe. The end of the catheter opposite the delivery end, referred to herein as the proximal end or the attachment end, is attached to a disclosed sonographic device. The contrast medium is generated by the actions of the sonographic device and the contrast medium is provided into and through the catheter(s) and out into the uterus. The desired amount of contrast medium is provided and visualization techniques are initiated, such as ultrasound or sonography, and can be used to visualize the movement of the contrast medium into the uterus, to visualize at least a portion of the structure of the uterus, for example, by providing the contrast medium, and/or to visualize entry, transit and/or exit of the contrast medium in at least one fallopian tube. If a fallopian tube is blocked, the contrast medium will not flow past the blockage but may flow to the contralateral tube if it is not blocked.

The disclosure herein refers to fluids such as air or saline, but it is contemplated that the present subject matter is not limited to air and/or saline, and that one of skill in the art can substitute air and/or saline for other appropriate fluids, such as other liquids, other gases or known contrast medium compositions. Methods of the present subject matter comprise making or generating a contrast medium (composition), and delivering a contrast medium to a body structure. A sonographic device of the present subject matter is used to make a contrast medium.

Disclosed herein are devices comprising two containers for fluids, such as air and saline. In an aspect, each container is a syringe body, also referred to as a syringe barrel or syringe container. The interior of each syringe barrel is traversed by a plunger. The plunger is moveable within the syringe barrel, from a proximal location in the barrel, wherein proximal refers to the end of the device closest to the operator and away from the fluid port of the device, to a distal location, wherein distal refers to the end of the device closest to the patient and nearer to the exit port, and from a distal location in the barrel to a proximal location in the barrel. A plunger may be comprised of a piston and a fluid seal having two surfaces, wherein the piston is attached to one surface of the fluid seal, the proximal surface, and the other surface, the distal surface, faces and contacts the deliverable fluid. A fluid seal, having two surfaces, forms a fluid seal within the container, so that a deliverable fluid is maintained or contained on the distal surface of the plunger surface (a first surface) and no deliverable fluid is present on proximal surface (a second surface). A deliverable fluid is the fluid contained within the container and which is intended to be used in making the contrast medium. As a plunger, comprising a piston and fluid seal, is moved through a syringe, there may be air or a slight vacuum created on the proximal side of the plunger, but there is no intention to provide the air on the proximal side of the seal, therefore this air is not a deliverable fluid. A liquid used in disclosed methods may be any of those disclosed herein, such as saline or water, or known contrast agent fluids. A gas may be any of those disclosed herein, such as air, carbon dioxide, oxygen, nitrogen or halocarbon compound gases, other gases, or known contrast agent gases.

A disclosed device generates and delivers a reproducible and reliable pattern of alternating air and fluid segments that is visible by detection methods such as sonography. The air/liquid pattern produced by a disclosed sonographic device is reproducible in that a substantially repeating pattern of alternating air segment and liquid segment is generated as a contrast medium composition by the sonographic device, and is provided to a body structure, and for example, is visible with a fallopian tube. The air/liquid contrast medium composition is consistently produced by a disclosed device and the contrast medium wherein the distance between the air/liquid interfaces is short enough and repeats regularly enough that movement of the composition through a structure is visible, such as by sonography. It is contemplated by the present disclosure that the distance between interfaces of a contrast medium of the present subject matter is not necessarily identical for every pair of interfaces but that the distances are sufficiently similar in length so as to form the perception of a repeating pattern of light and dark by detection means such as sonography, and that the structure of a body structure, such as fallopian tube, can be viewed by the movement of the regular pattern of light, and dark produced by the interfaces under detection means such as sonography. A liquid alone (without air) appears black when viewed by sonography as liquid reflects less sound echoes to the probe, therefore, long intervals of liquid may present a problem for the user to visualize a body structure, such as fallopian tube. Air appears white, as does bone, as air reflects more sound echoes to the probe, therefore, long intervals of air may be misinterpreted and easily confused with other body tissues, leading to uncertainty in the diagnosis, such as fallopian tube patency. Movement of the saline and air interfaces with a repeating pattern, as described by the current disclosure, that is frequent, regular, and alternating air/liquid, allows for an effect called reverberation that is caused by sound interfacing with two structures of sufficiently different reflective properties. With an erratic and not substantially regular pattern, the complexity of the sonohysterography procedure increases dramatically, making the reliability of the procedure questionable and the learning curve, for a medical professional to learn and perform the procedure, very steep. Specifically, erratic patterns consisting of small pattern frequencies creating a long segment of either air or saline can lead to misinterpretations as there will not appear to be movement of the contrast medium, and it is the perception of the movement of the contrast medium in and through a structure that is necessary for the medical professional to make an evaluation of the fallopian tubes. Visualization my be complicated by varying subject anatomical positions, such as of the uterus and fallopian tubes. Consistent movement of the frequent, substantially regular alternating pattern of a contrast medium, such as that generated by a disclosed device, will increase the likelihood of the body structure(s) being viewed by the medical professional to make the intended evaluation. As used herein, “viewing the contrast medium” may include viewing the contrast medium in the subject by healthcare professionals at the time of conducting the contrast medium procedure; or viewing the contrast medium in the subject by healthcare professionals after the time of conducting the contrast medium procedure because the images of the contrast medium in the subject were filmed, taped, transmitted by electronic means such as the internet, or captured in a manner known to those of skill in the art.

Methods of the present disclosure include methods for diagnosing structures of a subject. Such diagnostic methods may include, but are not limited to, diagnosing the physical condition of at least one of a subject's structures; diagnosing the patency of one or more fallopian tubes, structural aspects of one or more of the fallopian tubes or uterus, ovarian or abdominal structural aspects, or some or all of these. In a method of diagnosis, a sonographic device disclosed herein is used to make and deliver a visualizable contrast medium to one or more structures to be diagnosed. The one or more structures are visualized and a diagnosis is determined. A diagnosis may comprise one or more visualization procedures, and a visualization procedure may be preceded by or followed by pretreatment or post-treatment procedures. Pretreatment may comprise providing or administering anesthetic compounds or molecules to the subject, administering one or more therapeutic agents to a subject, A post treatment procedure may comprise using a sonographic device disclosed herein to provide a contrast medium comprising at least one therapeutic agent to a target site in a subject and contacting a structure or the target site with at least one therapeutic agent, and optionally, visualizing at least one structure of the subject.

In summary, a method of making a contrast medium, comprises, a) filling, simultaneously and at the same rate, both a liquid container and an air container, with liquid and air respectively, of a sonographic device disclosed herein, so that liquid enters through the fluid port and air enters through the air port, by moving each respective plunger in a proximal direction until the containers are filled; b) moving each respective plunger in a distal direction to move liquid and air from the respective containers, through respective air and liquid fluid connections to Y-shaped tubing to make a contrast medium that exits the fluid port.

In summary, a method of diagnosis by sonographic visualization of a body structure, comprises, a) filling, simultaneously and at the same rate, both a liquid container and an air container, with liquid and air respectively, of a sonographic device disclosed herein, so that fluid enters through the fluid port and air enters through the air port, by moving each respective plunger in a proximal direction until the containers are filled; b) attaching a catheter or catheter assembly to the fluid port c) optionally, positioning a delivery end of the catheter of catheter assembly at a target site in a subject; d) moving each respective plunger in a distal direction to move liquid and air from the respective containers, through respective air and liquid fluid connections to Y-shaped tubing to make a contrast medium that exits the fluid port and then exits the delivery end of the catheter or catheter assembly; viewing the contrast medium in the subject by sonography; and diagnosing the physical condition of the target site.

A method disclosed herein may comprise a) providing a disclosed sonographic device comprising a container assembly comprising at least one container for containing a liquid and one container for a gas or air, each container having a component, such as a plunger, for moving the respective liquid or air/gas to or from its respective container simultaneously and at the same rate, and fluid connections, such as tubing; b) filling each container with its liquid or air/gas respectively; c) moving the liquid and air/gas from its respective container, simultaneously and at the same rate (time and distance) to generate a contrast medium composition; d) providing the contrast medium composition to a body structure via a catheter comprising a catheter delivery end positioned in the same or a different body structure; and e) viewing the contrast medium composition in one or more body structures by ultrasound.

Methods of the present disclosure include methods for treating at least one structure of a subject with a composition comprising at least one therapeutic agent. Such treatment methods may include, but are not limited to, treatment of the uterus, treatment of one or more fallopian tubes, treatment of one or more of the fallopian tubes or the uterus, ovarian or abdominal structures, or some or all of these. In a method of treatment, a sonographic device disclosed herein is used to make and deliver a contrast medium comprising at least one therapeutic agent to one or more structures of a subject that is/are to be treated. The one or more structures may or may not be visualized, and the contrast medium composition comprising an effective amount of at least one therapeutic agent is applied to, or contacts, one or more structures of the subject, which may or may not be a target site, and the contact of the treatment contrast medium and the one or more structures comprises treatment of the one or more structures. A treatment method may comprise one or more visualization procedures, one or more treatment procedures, and visualization or treatment procedures may be preceded by or followed by pretreatment or post-treatment procedures. A pretreatment step may comprise using a sonographic device disclosed herein to provide a contrast medium to a target site in a subject and visualizing at least one structure of the subject.

In summary, a method of treatment of a body structure, comprises, a) filling, simultaneously and at the same rate, both a liquid container and an air container, with a liquid comprising an effective amount of at least one therapeutic agent, and air respectively, of a sonographic device disclosed herein, so that the liquid enters through the fluid port and air or gas enters through the air port, by moving each respective plunger in a proximal direction until the containers are filled; b) attaching a catheter or catheter assembly to the fluid port; c) optionally, positioning a delivery end of the catheter or catheter assembly at a target site in a subject; d) moving each respective plunger in a distal direction to move the liquid and the air or gas from the respective containers, through respective air/gas and liquid fluid connections to Y-shaped tubing to make a contrast medium that exits the fluid port into the catheter or catheter assembly; and then exits the delivery end of the catheter or catheter assembly; and treating at least one structure of the subject and optionally, viewing the contrast medium in the subject by sonography.

A contrast medium, also referred to herein as a contrast medium composition, may comprise a liquid and a gas, and optionally, surfactants, emulsifiers, or other stabilizing agents. The liquid, which may be seen as a carrier of the gas phase in the contrast medium composition, may be any liquid that is substantially free of solids and flows at normal or bodily temperatures. Contrast medium compositions may be made with a liquid that is flowable and forms a discrete liquid phase when in contact with a gas. For example, the liquid may be water or physiologically acceptable aqueous solutions including, but not limited to, physiological electrolyte solutions, physiological saline solutions, Ringer's solution or aqueous solutions of sodium chloride, calcium chloride, sodium bicarbonate, sodium citrate, sodium acetate, or sodium tartrate, glucose solutions, or solutions of mono- or polyhydric alcohol, e.g., ethanol, n-butanol, ethylene glycol, polyvinylpyrrolidone, or mixtures or combinations of these. Further, the liquid carrier may comprise physiologically acceptable non-aqueous solutions, including, but not limited to, anhydrous or substantially anhydrous carrier liquids, alcohols, glycols, polyglycols, synthetic perfluoranated hydrocarbons, or in mixtures or combination with other non-aqueous or aqueous liquids.

Contrast media compositions may comprise surfactants or compounds that stabilize the gas-liquid interface. Surfactants may be provided in the liquid phase or the gas phase of the contrast medium. For example, if a contrast medium comprises air and a liquid, such as saline, one or more surfactants may be added to the saline. Surfactants include tensides, such as lecithins; esters and ethers of fatty acids and fatty alcohols with polyoxyethylene and polyoxyethylated polyols like sorbitol, glycols and glycerol, cholesterol; and polyoxy-ethylene-polyoxypropylene polymers, viscosity raising and stabilizing compounds, mono- and polysaccharides (glucose, lactose, sucrose, dextran, sorbitol); polyols, e.g., glycerol, polyglycols; and polypeptides like proteins, gelatin, oxypolygelatin, plasma protein, amphipathic compounds capable of forming stable films in the presence of water and gases, such as the lecithins (phosphatidyl-choline) and other phospholipids, inter alia phosphatidic acid (PA), phosphatidylinositol, phosphatidylethanolamine (PE), phosphatidylserine (PS), phosphatidylglycerol (PG), cardiolipin (CL), sphingomyelins, the plasmogens, the cerebrosides, natural lecithins, such as egg lecithin or soya bean lecithin, or synthetic lecithins such as saturated synthetic lecithins, for example, dimyristoylphosphatidylcholine, dipalmitoylphosphatidylcholine or distearoylphosphatidylcholine or unsaturated synthetic lecithins, such as dioleylphosphatidylcholine or dilinoleylphosphatidylcholine, free fatty acids, esters of fatty acids with polyoxyalkylene compounds like polyoxypropylene glycol and polyoxyalkylene glycol; ethers of fatty alcohols with polyoxyalkylene glycols; esters of fatty acids with polyoxyalkylated sorbitan; soaps; glycerol-polyalkylene stearate; glycerolpolyoxyethylene ricinoleate; homo- and copolymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-di- and triglycerides of saturated or unsaturated fatty acids; glycerides of soya-oil and sucrose, block copolymers of polyoxypropylene and polyoxyethylene (poloxamers) polyoxyethylenesorbitans, sorbitol, glycerol-polyalkylene stearate, glycerolpolyoxyethylene ricinoleate, homo- and copolymers of polyalkylene glycols, soybean-oil as well as hydrogenated derivatives, ethers and esters of sucrose or other carbohydrates with fatty acids, fatty alcohols, glycerides of soya-oil, dextran, sucrose and carbohydrates. Surfactants may be film forming and non-film forming and may include polymerizable amphiphilic compounds of the type of linoleyl-lecithins or polyethylene dodecanoate, phosphatidic acid, phosphatidylcholine, phosphatidylethanolamine, phosphatidylserine, phosphatidylglycerol, phosphatidylinositol, cardiolipin, sphingomyelin and biocompatible and amphipathic compound capable of forming stable films in the presence of an aqueous phase and a gas, phospholipids including phosphatidylcholine (PC) with both saturated and unsaturated lipids; including phosphatidylcholine such as dioleylphosphatidylcholine; dimyristoylphosphatidylcholine (DMPC), dipentadecanoylphosphatidylcholine-, dilauroylphosphalidylcholine (DLPC), dipalmitoylphosphatidylcholine (DPPC); disteracylphosphatidylcholine (DSPC); and diarachidonylphosphatid-ylcholine (DAPC); phosphatidylethanolamines (PE), such as dioleylphosphatidylethanolamine, dipaimitoylphosphatidylethanolamine (DPPE) and distearoylphosphatidylethanolamine (DSPE); phosphatidylserine (PS) such as dipalmitoyl phosphatidylserine (DPPS), disteraoylphosphatidylscrine (DSPS); phosphatidylglycerols (PG), such as dipalmitoylphosphatidylglycerol (DPPG), distearoylphosphatidylglycerol (DSPG); and phosphatidylinositol. Combination and mixtures of surfactants, emulsifiers, or other stabilizing agents may also be used. Surfactants, emulsifiers, or other stabilizing agents may aerosolized within the gas phase.

Contrast medium compositions may comprise gases, and any physiologically acceptable gas may be used in contrast medium compositions. The term “gas” as used herein includes any substances (including combinations and mixtures) substantially in gaseous form at the normal human body (37° C.) temperature. Close to 200 different gases have been identified as potentially useful for making ultrasound contrast agents, and include oxygen, air, nitrogen, carbon dioxide or mixtures thereof, helium, argon, xenon, krypton, CHClF₂ or nitrous oxide, sulfur hexafluoride, tetrafluoromethane, chlorotrifluoromethane, dichlorodifluoromethane, bromotrifluoromethane, bromochlorodifluoromethane, dibromodifluoromethane dichlorotetrafluoroethane, chloropentafluoroethane, hexafluoroethane, hexafluoropropylene, octafluoropropane, hexafluoro-butadiene, octafluoro-2-butene, octafluorocyclobutane, decafluorobutane, perfluorocyclopentane, dodecafluoropentane, fluorinated gases including materials which contain at least one fluorine atom such as SF₆, freons (organic compounds containing one or more carbon atoms and fluorine, i.e. CF₄, C₂F₆, C₃F₈, C₄F₈, C₄F₁₀, CBrF₃, CCl₂F₂, C₂ClF₃ and CBrClF₂ and perfluorocarbons. The term perfluorocarbon refers to compounds containing only carbon and fluorine atoms and includes saturated, unsaturated, and cyclic perfluorocarbons such as perfluoroalkanes such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes and perfluoroheptanes; perfluoroalkenes such as perfluoropropene, perfluorobutenes (e.g. perfluorobut-2ene) and perfluorobutadiene; perfluoroalkynes such as perfluorobut-2-yne, and perfluorocycloalkanes such as perfluorocyclobutane, perfluoromethylcyclobutane, perfluorodimethylcyclobutanes, perfluorotrimethylcyclobutanes, perfluorocyclopentane, perfluoromethycylopentane, perfluorodimethylcyclopentanes, perfluorocyclohexane, perfluoromethylcyclohexane and perfluorocycloheptane). The saturated perfluorocarbons, which are usually preferred, have the formula CₙFₙ₊₂, where n is from 1 to 12, from 2 to 10, from 3 to 8 or from 3 to 6. Suitable perfluorocarbons include, for example, CF₄, C₂F₆, C₃F₈, C₄F₈, C₄F₁₀, C₅F₁₂, C₆F₁₂, C₇F₁₄, CgF₁₈, and CgF₂O.

Diagnostic or therapeutic methods are disclosed that comprise making and delivering compositions, such as contrast medium compositions or contrast medium compositions comprising an effective amount of at least one therapeutic agent, made by a disclosed sonographic device, wherein such contrast medium compositions comprise at least one therapeutic agent, which may also be called a treatment contrast medium, and the treatment contrast medium is provided to a structure using a disclosed sonographic device and optionally, a catheter or a catheter assembly as described herein. For example, a treatment contrast medium composition comprising an effective amount of at least one therapeutic agent may be provided to a fallopian tube. Therapeutic agents include, but are not limited to, anesthetics, methotrexate; androgenic or estrogenic hormones, antiandrogens; fertility enhancing compounds; fertility interfering compounds; motility enhancing compounds; motility interfering compounds; compounds affecting the cilia/deciliation cycle in a fallopian tube; cilia growth enhancing or interfering compounds; ovarian follicle treatment compounds; antibacterial compounds or molecules; antimicrobial compounds or molecules; antifungal compounds or molecules; antiviral compounds or molecules; antimycoplasmal compounds or molecules; or antiparisital compounds or molecules; compounds or molecules that reduce inflammation or scar tissue formation; mucoproteins; electrolytes or enzymes to enhance or inhibit fertility; progesterone; estrogen; estrogen-like molecules including, but not limited to, estradiol, oestrogen, estrone, estriol, oestrone, oestriol, oestradiol, 17β-estradiol, and estra-1,3,5(10)-triene-3,17β-diol; adrenergic active compounds or molecules; noradrenergic active compounds or molecules; nonsteroidal anti-inflammatory drug; prostaglandins; compounds or molecules that may treat or prevent conditions related to the fallopian tube, uterus, ovaries, or other organs or coverings reached by a composition flowing from the cornua or ostia of a fallopian tube through the fallopian tube and out the opening at the fimbria; or combinations thereof. Treatment contrast agent compositions may comprise therapeutic agents comprising hormones for fertility; fertility enhancing compounds or molecules; gametes; sperm; ova; combinations of sperm and ova; one or more zygotes; or one or more embryos; or combinations thereof. In methods for delivery of treatment contrast medium compositions to a subject's structure(s), a treatment contrast medium composition may comprise a treatment contrast medium composition comprising a gas (which may or may not comprise one or more therapeutic agents) with a liquid comprising and effective amount of at least one therapeutic agent, and the delivery of the treatment contrast medium may be monitored by visualization techniques such as ultrasound. A treatment method, comprising a treatment contrast medium composition comprising one or more therapeutic agents that is visualizable, made and delivered by a sonographic device disclosed herein, may provide both treatment and diagnosis of the condition of a structure in one step of making and delivering the treatment contrast medium composition. A treatment method, comprising making and delivering a visualizable treatment contrast medium composition comprising an effect amount of one or more therapeutic agents, made and delivered by a sonographic device disclosed herein, may be employed to limit or locate the at least one therapeutic agent at the targeted structure via sonographic imaging. The sonographic imaging may be in sequence, by for example, using a diagnostic contrast medium (with no therapeutic agent) or the treatment contrast medium, comprising air or gas, may be visualized while the treatment contrast medium is being delivered to one or more structures of the subject.

In an aspect, a method may comprise use of a sonographic device disclosed herein with known hysterosalpingography procedures. For example, such procedures may be performed prior to or after use of a sonographic device disclosed herein. A procedure may comprise providing saline only to the uterine cavity to at least in part fill the uterus or to distend the uterus. The uterus may be visualized by detection methods, or not. The saline is then released from the uterus, such as by releasing a balloon used to seal the uterus closed from the cervix, or by withdrawing the catheter that provided the saline to the uterus. Alternatively, the saline may flow out of the fallopian tubes. After such a pre-treatment procedure, a contrast medium device of the present subject matter may be used by attaching the device to a catheter having its delivery end within the uterus, generating a contrast medium composition and providing the contrast medium composition to the uterus and optionally, to at least one fallopian tube. Post-treatments may also be provided to the uterus or fallopian tubes after providing the contrast medium composition. For example, a therapeutic composition or an embryo composition may then be provided to the uterus or fallopian tube. Though not wishing to be bound by any particular theory, it is theorized that providing a contrast medium composition via a sonographic device disclosed herein, aids the subject in later becoming pregnant. It is thought that there is a higher incidence of becoming pregnant is found in women who have undergone a procedure comprising using a sonographic device disclosed herein and air/saline contrast medium composition disclosed. The present subject matter comprises a method for enhancing pregnancy in a female, aiding in or obtaining a pregnant condition in a female, or increasing the fertility of a female, comprising, providing a sonographic device disclosed herein comprising a container assembly comprising at least one container for containing a liquid and one container for a gas or air, each container having a component, such as a plunger, for moving the respective liquid or air/gas to or from its respective container simultaneously and at the same rate, and fluid connections, such as tubing; b) filling each container with its liquid or air/gas respectively; c) moving the liquid and air/gas from its respective container, simultaneously and at the same rate (time and distance) to generate a contrast medium composition; d) providing the contrast medium composition to a body structure via a catheter comprising a catheter delivery end positioned in the same or a different body structure; and e) viewing the contrast medium composition in one or more body structures by ultrasound. Visualization by detection methods such as sonography may or may not be performed.

In methods disclosed herein, one or both fallopian tubes may be viewed simultaneously, sequentially or in separate procedures. In some instances, it may not be possible to view both fallopian tubes in the same plane of sonographic imaging. One or both fallopian tubes may not fill simultaneously, for example, a spasm may constrict the opening or a portion of a fallopian tube. If fallopian tubes are to be examined, the contrast medium may be delivered to the uterine cornua or at the opening of the fallopian tube by a catheter or catheter assembly. In an aspect, a sufficient amount of contrast medium may be provided to the uterus and fallopian tubes so that both the uterus and fallopian tubes may be assessed by visualization techniques. Complete uterine cavity distension may not be required to assess one or both fallopian tubes.

Disclosed herein are methods comprising ultrasound to visualize the contrast medium within a structure. Often, procedures performed with ultrasound use a transvaginal probe where the probe can be placed closer to the fallopian tubes. Positioning of the probe to achieve a sagittal view allows for visualization of the sonographic imaging agent in the uterine cavity with the catheter in place, verifying forward flow into the uterine cavity absent retrograde flow back towards the vagina. Positioning of the probe to achieve a transverse view allows for visualization of the sonographic imaging agent from the uterine cavity into the fallopian tubes, which may result viewing both fallopian tubes or each fallopian tube in a certain plane.

The present disclosure contemplates that a structure that is viewable using a contrast medium may be viewed using the contrast medium compositions of the present subject matter, made and delivered by a sonographic device disclosed herein. For example, a structure to be visualized is at least one fallopian tube of a human or animal.

The present disclosure comprises systems comprising a disclosed sonographic device, and optionally a catheter or catheter assembly for providing the contrast medium to a subject. A system may further comprise a syringe for inflating a catheter balloon. For example, a syringe may comprise a variably locking syringe, such as that described in U.S. patent application Ser. No. 18/437,667, which is herein incorporated in its entirety for all of its teachings. A system may further comprise a separate individual container (e.g., a bowl) for holding fluid used in making the contrast medium. A system comprises simultaneously, and at the same rate, filling both the fluid and air containers (e.g., syringes) of the sonographic device by moving the sonographic device plungers in a proximal direction, optionally with fluid provided in the separate container. After filling both sonographic device containers, contrast medium is made by moving the sonographic device plungers simultaneously, and at the same rate, in a distal direction to move liquid and air to form the contrast medium, which is provided to the subject or to a catheter or catheter assembly. A variably locking syringe, is used to expand, or deflate, a catheter balloon, such as a balloon on the catheter that prevents retrograde flow of the contrast medium from the uterus. The present disclosure contemplates visualization methods known or used in the art, including, but not limited to, sonography and fluorography.

The present disclosure comprises one or more kits. The present disclosure comprises a kit comprising a disclosed sonographic device. The present disclosure comprises a kit comprising a disclosed sonographic device, and instructions for use of the sonographic device. The present disclosure comprises a kit comprising a disclosed sonographic device, and optionally a catheter or catheter assembly for providing contrast medium to a subject. The present disclosure comprises a kit comprising a disclosed sonographic device, and optionally a catheter or catheter assembly for providing contrast medium to a subject, and instructions for use of the disclosed sonographic device, and optionally, use of the catheter or catheter assembly. A kit disclosed herein may further comprise a syringe for inflating/deflating a catheter balloon. For example, a syringe may be a variably locking syringe, such as that described in U.S. patent application Ser. No. 18/437,667, which is herein incorporated in its entirety for all of its teachings. A kit disclosed herein may comprise a sonographic device, and a syringe, for example, a variably locking syringe. A kit disclosed herein may comprise a sonographic device, a syringe, for example, a variably locking syringe, and instructions for use of the sonographic device and the syringe, for example, the variably locking syringe. A kit may further comprise a separate individual container (e.g., a bowl) for holding fluid used in making the contrast medium. A kit may comprise a disclosed sonographic device, a catheter or catheter assembly for providing contrast medium to a subject, a syringe, for example, a variably locking syringe, a separate container for liquid, and instructions for use of the components of the kit. A kit may comprise a disclosed sonographic device, a catheter or catheter assembly for providing contrast medium to a subject, a variably locking syringe, a separate container for liquid, and instructions for use of the components of the kit. A kit may comprise a disclosed sonographic device, a catheter or catheter assembly for providing contrast medium to a subject, a variably locking syringe, and instructions for use of the components of the kit.

### EXAMPLES

Some non-limiting examples of the present subject matter are provided as follows:

Example 1 is a sonographic device for creating and delivering contrast medium, including: a first syringe body for containing a liquid, the first syringe body having a first plunger positioned therein; a second syringe body for containing a gas, the second syringe body having a second plunger positioned therein; liquid connecting tubing in fluid connection with the first syringe body; air connecting tubing in fluid connection with the second syringe body; Y-shaped tubing in fluid connection with both the liquid connecting tubing and the air connecting tubing; an air inlet port in fluid connection with the second syringe body; a fluid port in fluid connection with both syringe bodies through the Y-shaped tubing; a first one-way valve positioned between the air inlet port and the second syringe body; and a second one-way valve positioned between the second syringe body and the Y-shaped tubing; wherein the device is configured such that both syringe bodies fill simultaneously and at a same rate when the first and second plungers are moved proximally, and when the first and second plungers are moved distally contrast medium composition including alternating segments of liquid and gas exits the fluid port as the first fluid.

Example 2 is the sonographic device of Example 1, further including a casing defining an enclosed portion, a connecting portion, and a handle, wherein the first syringe body and second syringe body are positioned within the casing.

Example 3 is the sonographic device of Example 2, wherein the casing includes slots formed in the handle through which syringe flanges of the first and second syringe bodies protrude.

Example 4 is the sonographic device of Example 2, further including a syringe plunger actuator operatively connected to both the first plunger and the second plunger, wherein the syringe plunger actuator is configured to move both plungers simultaneously and at the same rate.

Example 5 is the sonographic device of Example 2, wherein the connecting portion includes first and second components that are joined together at connection sites.

Example 6 is the sonographic device of Example 1, wherein the fluid port includes an attachment element for connecting to a catheter or catheter assembly.

Example 7 is the sonographic device of Example 1, further including air inlet connecting tubing and a tubing connection positioned between the first one-way valve and the air connecting tubing.

Example 8 is the sonographic device of Example 1, further including air exiting connecting tubing positioned between the second one-way valve and the Y-shaped tubing.

Example 9 is the sonographic device of Example 1, wherein the first one-way valve has a cracking pressure of approximately from 0.014 to 0.087 lb/in².

Example 10 is the sonographic device of Example 1, wherein the second one-way valve has a cracking pressure of approximately from 0.014 to 0.087 lb/in².

Example 11 is the sonographic device of Example 1, wherein both the first one-way valve and the second one-way valve have a substantially similar cracking pressure of approximately from 0.014 to 0.087 lb/in².

Example 12 is the sonographic device of Example 1, wherein the first one-way valve has a higher cracking pressure than the second one-way valve.

Example 13 is the sonographic device of Example 1, wherein the second one-way valve has a cracking pressure of approximately less than 0.087 lb/in² such that the valve opens at low pressure to allow gas to flow quickly through from a start of movement of the plungers.

Example 14 is the sonographic device of Example 13, wherein at the cracking pressure, the second one-way valve is completely open and gas flows freely through the valve at the pressure provided by distal movement of the second plunger.

Example 15 is the sonographic device of Example 1, wherein the liquid connecting tubing has a larger diameter than the air connecting tubing.

Example 16 is the sonographic device of Example 15 wherein a diameter of the liquid connecting tubing and a diameter than the air connecting tubing are selected to equalize rates of filling both syringe bodies.

Example 17 is a method of creating and delivering contrast medium using a sonographic device including a first container for containing a liquid, a second container for containing a gas, each container having a plunger positioned therein, and fluid connection tubing connecting both containers to a fluid port, the method including: filling both the first container and the second container simultaneously and at a same rate by moving both plungers proximally until the containers are filled with liquid and gas respectively; and delivering contrast medium composition by moving both plungers distally simultaneously and at the same rate to move the liquid and gas from the respective containers through the fluid connection tubing to create the contrast medium composition including alternating segments of liquid and gas, such that the contrast medium composition exits with little or no unmixed liquid preceding the contrast medium composition.

Example 18 is the method of Example 17, further including attaching a catheter to the fluid port and positioning a delivery end of the catheter at a target site in a subject before delivering the contrast medium composition.

Example 19 is the method of Example 18, wherein the target site includes at least one of a uterus or a fallopian tube.

Example 20 is the method of Example 17, further including viewing the contrast medium composition in a subject by sonography.

Example 21 is the method of Example 20, further including determining patency or occlusion of at least one fallopian tube based on sonographic viewing.

Example 22 is the method of Example 17, wherein the liquid includes saline and the gas includes air.

Example 23 is the method of Example 17, wherein the contrast medium composition includes a therapeutic agent.

Example 24 is the method of Example 23, further including treating at least one structure of a subject by contacting the structure with the contrast medium composition including the therapeutic agent.

Example 25 is the method of Example 17, wherein filling both containers simultaneously and at the same rate includes drawing liquid through the fluid port and drawing gas through an air inlet port.

Example 26 is the method of Example 17, wherein the sonographic device includes a first one-way valve positioned between an air inlet port and the second container, and a second one-way valve positioned between the second container and Y-shaped tubing.

Example 27 is the method of Example 26, wherein the first one-way valve and second one-way valve each have a cracking pressure of approximately from 0.014 to 0.087 lb/in².

Example 28 is the method of Example 17, wherein the contrast medium composition creates a substantially repeating pattern of alternating air segments and liquid segments that is reproducible and visible by sonographic detection.

The above examples may include elements in addition to those shown or described. The foregoing examples are not intended to be an exhaustive or exclusive list of examples and variations of the present subject matter.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, when a compound is referred to as a monomer or a compound, it is understood that this is not interpreted as one molecule or one compound. For example, two monomers generally refers to two different monomers, and not two molecules.

As used herein, the terms “optional” or “optionally” means that the subsequently described event or circumstance can or cannot occur, and that the description includes instances where said event or circumstance occurs and instances where it does not.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, a mammalian subject is a human. A patient refers to a subject afflicted with a disease or disorder or requiring contraception. The term “patient” includes human and veterinary subjects.

As used herein, the terms “administering” and “administration” refer to any method of providing a disclosed composition to a subject.

As used herein, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Optional additives as defined herein, at a level that is appropriate for such additives, and minor impurities are not excluded from a composition by the term “consisting essentially of”.

When a composition, a process, a structure, or a portion of a composition, a process, or a structure, is described herein using an open-ended term such as “comprising,” unless otherwise stated the description also includes an embodiment that “consists essentially of” or “consists of” the elements of the composition, the process, the structure, or the portion of the composition, the process, or the structure.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

The term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

The term “or”, as used herein, is inclusive; that is, the phrase “A or B” means “A, B, or both A and B”. More specifically, a condition “A or B” is satisfied by any one of the following: A is true (or present) and Bis false (or not present); A is false (or not present) and B is true (or present); or both A and B are true (or present). Exclusive “or” is designated herein by terms such as “either A or B” and “one of A or B”, for example.

In addition, the ranges set forth herein include their endpoints unless expressly stated otherwise. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. The scope of the invention is not limited to the specific values recited when defining a range.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that will have become recognized in the art as suitable for a similar purpose.

Unless stated otherwise, all percentages, parts, ratios, and like amounts, are defined by weight.

All patents, patent applications and references included herein are specifically incorporated by reference in their entireties for their teachings.

It should be understood, of course, that the foregoing relates only to embodiments of the present disclosure and that numerous modifications or alterations may be made therein without departing from the spirit and the scope of the disclosure as set forth in this disclosure.

The present disclosure is further illustrated by the examples contained herein, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

Although the exemplary embodiments of the present subject matter describe in detail methods, syringe assembly devices, delivery systems, and compositions, the present invention is not limited to these embodiments. There are numerous modifications or alterations that may suggest themselves to those skilled in the art for use of the methods, delivery systems, and compositions herein for the diagnosis or treatment of a variety of conduits in both human and non-human mammals.

The present subject matter is further illustrated by way of the examples contained herein, which are provided for clarity of understanding. The exemplary embodiments should not be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

1. A sonographic device for creating and delivering contrast medium, comprising:
a first syringe body for containing a liquid, the first syringe body having a first plunger positioned therein;
a second syringe body for containing a gas, the second syringe body having a second plunger positioned therein;
liquid connecting tubing in fluid connection with the first syringe body;
air connecting tubing in fluid connection with the second syringe body;
Y-shaped tubing in fluid connection with both the liquid connecting tubing and the air connecting tubing;
an air inlet port in fluid connection with the second syringe body;
a fluid port in fluid connection with both syringe bodies through the Y-shaped tubing;
a first one-way valve positioned between the air inlet port and the second syringe body; and
a second one-way valve positioned between the second syringe body and the Y-shaped tubing;
wherein the device is configured such that both syringe bodies fill simultaneously and at a same rate when the first and second plungers are moved proximally, and when the first and second plungers are moved distally contrast medium composition comprising alternating segments of liquid and gas exits the fluid port as the first fluid.

2. The sonographic device of claim 1, further comprising a casing defining an enclosed portion, a connecting portion, and a handle, wherein the first syringe body and second syringe body are positioned within the casing.

3. The sonographic device of claim 2, wherein the casing comprises slots formed in the handle through which syringe flanges of the first and second syringe bodies protrude.

4. The sonographic device of claim 2, further comprising a syringe plunger actuator operatively connected to both the first plunger and the second plunger, wherein the syringe plunger actuator is configured to move both plungers simultaneously and at the same rate.

5. The sonographic device of claim 2, wherein the connecting portion comprises first and second components that are joined together at connection sites.

6. The sonographic device of claim 1, wherein the fluid port comprises an attachment element for connecting to a catheter or catheter assembly.

7. The sonographic device of claim 1, further comprising air inlet connecting tubing and a tubing connection positioned between the first one-way valve and the air connecting tubing.

8. The sonographic device of claim 1, further comprising air exiting connecting tubing positioned between the second one-way valve and the Y-shaped tubing.

9. The sonographic device of claim 1, wherein the first one-way valve has a cracking pressure of approximately from 0.014 to 0.087 lb/in2.

10. The sonographic device of claim 1, wherein the second one-way valve has a cracking pressure of approximately from 0.014 to 0.087 lb/in2.

11. The sonographic device of claim 1, wherein both the first one-way valve and the second one-way valve have a substantially similar cracking pressure of approximately from 0.014 to 0.087 lb/in2.

12. The sonographic device of claim 1, wherein the first one-way valve has a higher cracking pressure than the second one-way valve.

13. The sonographic device of claim 1, wherein the second one-way valve has a cracking pressure of approximately less than 0.087 lb/in2 such that the valve opens at low pressure to allow gas to flow quickly through from a start of movement of the plungers.

14. The sonographic device of claim 13, wherein at the cracking pressure, the second one-way valve is completely open and gas flows freely through the valve at the pressure provided by distal movement of the second plunger.

15. The sonographic device of claim 1, wherein the liquid connecting tubing has a larger diameter than the air connecting tubing.

16. The sonographic device of claim 15 wherein a diameter of the liquid connecting tubing and a diameter than the air connecting tubing are selected to equalize rates of filling both syringe bodies.

17. A method of creating and delivering contrast medium using a sonographic device including a first container for containing a liquid, a second container for containing a gas, each container having a plunger positioned therein, and fluid connection tubing connecting both containers to a fluid port, the method comprising:
filling both the first container and the second container simultaneously and at a same rate by moving both plungers proximally until the containers are filled with liquid and gas respectively; and
delivering contrast medium composition by moving both plungers distally simultaneously and at the same rate to move the liquid and gas from the respective containers through the fluid connection tubing to create the contrast medium composition comprising alternating segments of liquid and gas, such that the contrast medium composition exits with little or no unmixed liquid preceding the contrast medium composition.

18. The method of claim 17, further comprising attaching a catheter to the fluid port and positioning a delivery end of the catheter at a target site in a subject before delivering the contrast medium composition.

19. The method of claim 18, wherein the target site comprises at least one of a uterus or a fallopian tube.

20. The method of claim 17, further comprising viewing the contrast medium composition in a subject by sonography.

21. The method of claim 20, further comprising determining patency or occlusion of at least one fallopian tube based on sonographic viewing.

22. The method of claim 17, wherein the liquid comprises saline and the gas comprises air.

23. The method of claim 17, wherein the contrast medium composition comprises a therapeutic agent.

24. The method of claim 23, further comprising treating at least one structure of a subject by contacting the structure with the contrast medium composition comprising the therapeutic agent.

25. The method of claim 17, wherein filling both containers simultaneously and at the same rate comprises drawing liquid through the fluid port and drawing gas through an air inlet port.

26. The method of claim 17, wherein the sonographic device comprises a first one-way valve positioned between an air inlet port and the second container, and a second one-way valve positioned between the second container and Y-shaped tubing.

27. The method of claim 26, wherein the first one-way valve and second one-way valve each have a cracking pressure of approximately from 0.014 to 0.087 lb/in2.

28. The method of claim 17, wherein the contrast medium composition creates a substantially repeating pattern of alternating air segments and liquid segments that is reproducible and visible by sonographic detection.
